(19) United States
(12) Patent Application Publication
(10) Pub. No.: US 2002/0071864 A1
(43) Pub. Date: Jun. 13, 2002

(54) Rapidly disintegrable tablet for oral administration

(75) Inventors: Hyun-Soo Kim, Kunpo-si (KR); Young-Joon Park, Kwachun-si (KR); Dae-Sik Kang, Ansan-si (KR)

Correspondence Address: SHAHAN ISLAM, ESQ., ROSENMAN & COLIN LLP, 575 Madison Avenue, New York, NY, 10022-2585, US

(73) Assignee: Yuhan Corporation, Seoul (KR)

(21) Appl. No.: 10/016,821

(22) Filed: Dec. 7, 2001

Related U.S. Application Data
(63) Continuation-in-part of application No. 09/536,163, filed on Mar. 25, 2000.

(30) Foreign Application Priority Data
Mar. 25, 1999 (KR) 1999-10172

(51) Int. Cl. A61K 9/20

(52) U.S. Cl. 424/464

(57) ABSTRACT

The present invention relates to a rapidly disintegrable tablet for oral administration, which disintegrates in the oral cavity within 60 seconds, consisting essentially of (i) a therapeutically effective amount of an active ingredient, (ii) spray-dried mannitol, of which at least 80% has an average particle size over 100 μm, (iii) crospovidone, and (iv) one or more pharmaceutically acceptable excipients, the tablet containing no microcrystalline cellulose.

### CROSS REFERENCE TO RELATED APPLICATION

This application is a continuation-in-part (CIP) application of U.S. Ser. No. 09/536,163 filed on Mar. 25, 2000, which is now abandoned and claims priority thereon pursuant to 35 USC section 120.

### FIELD OF THE INVENTION

The present invention relates to a rapidly disintegrable tablet formulation of a drug, and more particularly, to a drug tablet for oral administration, which disintegrates rapidly in the oral cavity, comprising a therapeutically effective amount of an active ingredient, spray-dried mannitol as a disintegrant, crospovidone as a co-disintegrant, and one or more pharmaceutically acceptable excipients, without microcrystalline cellulose.

### BACKGROUND OF THE INVENTION

It is not feasible to orally administer a conventional drug tablet to those having deglutition difficulties, or to patients whose water-intake must be restrictive. Therefore, liquid type formulations are usually prescribed for those people, but liquid formulations have the problems of low storage stability, handling difficulties and the inconvenience in measuring an accurate dose. Accordingly, there have been efforts to develop a rapidly disintegrable tablet formulation, which disintegrates rapidly and converts into a liquid form by the action of saliva in the oral cavity.

U.S. Pat. Nos. 4,371,516, 5,501,816 and 5,720,974 disclose processes for the preparation of porous, rapidly disintegrable tablets, which include the steps of adding a small quantity of a solvent to sugars, alcohols or carbohydrates to obtain a tablet mixture and removing the solvent therefrom. However, these processes have low productivity due to the involvement of complicated process steps and the tablets obtained thereby are easily friable and do not meet the hardness required for withstanding breakage during commercial handling.

U.S. Pat. No. 5,464,632 and European Patent Publication No. 839,526 also disclose rapidly disintegrable tablets, which comprise one or more disintegrants including microcrystalline cellulose and swelling agents. However, the water-insoluble microcrystalline cellulose remains undissolved in the oral cavity for some time, which often provides irritating sensation to patients.

Further, U.S. Pat. Nos. 5,958,453 relates to a buccal disintegration or dissolution type solid pharmaceutical preparation comprising a three-component adjuvant of erythritol, crystalline cellulose, and crospovidone. However, this preparation has a disadvantage of poor organoleptic feel.

U.S. Pat. No. 6,024,981, on the other hand, discloses a hard, compressed, rapidly dissolvable oral dosage form comprising a matrix including a non-direct-compression filler and a lubricant, said dosage form having a friability of about 2% or less when tested according to the U.S. Patent, and having a hardness of at least about 15 N (Newton). This patent is characterized in that a conventional non-direct compressing matrix is mixed with a large amount of a lubricant so as to provide a dosage with the specified properties, thus making it possible to directly compress the dosage using lower than expected compression forces.

Japanese Patent No. sho61-85330 discloses an excipient for direct tableting, which is obtained by spray-drying an aqueous solution of D-mannitol at 120 to 140° C. However, although this patent disclosed spray-dried mannitol has improved fluidity and disintegration properties, there is no mention of improved dissolution rate, which is essentially required to secure organoleptic feel appropriate to a rapidly disintegrable tablet in the oral cavity.

The present inventors have endeavored to develop an improved rapidly disintegrable tablets by solving the aforementioned problems; and, have discovered that a tablet comprising spray-dried mannitol having a specified particle size range and crospovidone, a cross-linked poly(N-vinyl-2-pyrrolidinone), disintegrates rapidly in the oral cavity, leaving no unpleasant water-insoluble residues, and has a hardness such that it is not friable during handling or shipment.

### SUMMARY OF THE INVENTION

Accordingly, it is an object of the present invention to provide an improved rapidly disintegrable tablet for oral administration comprising a pharmacologically active ingredient, spray-dried mannitol and crospovidone. In accordance with the present invention, there is provided a tablet for oral administration, which disintegrates in the oral cavity within 60 seconds, consisting essentially of (i) a therapeutically effective amount of an active ingredient, (ii) spray-dried mannitol, of which at least 80% has an average particle size over 100 μm, (iii) crospovidone and (iv) one or more pharmaceutically acceptable excipients, the tablet containing no microcrystalline cellulose.

### DETAILED DESCRIPTION OF THE INVENTION

As used herein, the term “therapeutically effective amount” of an active ingredient refers to the amount which produces the desired therapeutic response upon oral administration and can be readily determined by one skilled in the art. In determining the therapeutically effective amount, a number of factors are considered, including but not limited to: the particular compound administered, the bioavailability characteristics of the pharmaceutical composition administered, the dose regimen selected, and other relevant factors.

There is no limitation to the pharmacologically active ingredient to be used in the present invention. Examples of the pharmacologically active ingredient, which may be used in the present invention, are gastrointestinal function conditioning agents, anti-inflammatory agents, analgesics, agents for erectile dysfunction therapy, anti-migraines, anti-cholinergic agents, antihistaminic agents, cardiovascular agents, diuretics, anti-hypertensive agents, anti-hypolipidemic agents, anti-ulcer agents, anti-emetics, anti-asthmatic agents, anti-depressants, vitamins, anti-thrombotic agents, chemotherapeutic agents, hormones, anthelmintic agents and anti-diabetic agents.

Representative examples of the above-mentioned gastrointestinal function conditioning agents include bromopride, metoclopramide, cisapride and domperidone; the anti-inflammatory agents, aceclofenac, diclofenac, flubiprofen, sulindac and celecoxib; the analgesics, acetaminophen and aspirin; the agents for erectile dysfunction therapy, sildenafil and apomorphine; the anti-migraines, sumatriptan and ergotamin; anti-cholinergic agents, scopolamine hydrobromide; the antihistaminic agents, loratadine, fexofenadine and cetirizine; the cardiovascular agents, nitroglycerine and isosorbide dinitrate; the diuretics, furocemide and spironolactone; the anti-hypertensive agents, propranolol, amlodipine, felodipine, nifedipine, captoprile, ramiprile, atenolol and diltiazem; the anti-hypolipidemic agents, simvastatin, atrovastatin and pravastatin; the anti-ulcer agents, cimetidine, ranitidine, famotidine, omeprazole and lansoprazol; the anti-emetics, meclizine hydrochloride, ondansetron hydrochloride, granisetron, ramosetron and tropisetron; the anti-asthmatic agents, aminophylline, theophylline, terbutaline, fenoterol, formoterol and ketotifen; the anti-depressants, fluoxetine and sertraline; the vitamins, Vit B1, B2, B6, B12 and C; the anti-thrombotic agents, sulfinpyrazone, dipyridamole and ticlopidine; the chemotherapeutic agents, cefaclor, bacampicillin, sulfamethoxazole and rifampicin; the hormones, dexamethasone and methyltestosterone; the anthelmintic agents, piperazine, ivermectine and mebendazole; and the anti-diabetic agents, acarbose, gliclazid and glipizid.

Preferable active ingredients, which may be used in the present invention, include acetaminophen, domperidone, famotidine, meclizine hydrochloride, scopolamine hydrobromide, ondansetron hydrochloride, cisapride, granisetron, sildenafil, loratadine, and amlodipine.

The spray-dried mannitol used as a primary disintegrant in the inventive tablet may be prepared by spray-drying an aqueous solution of crystalline mannitol and it comprises one having an average particle size over 100 μm in an amount of at least 80%.

A commercially available spray-dried mannitol powder (e.g., PEARLITOL SD 200®, Roquette, France), having the said average particle size, may also be used in the present invention.

A spray-dried mannitol powder dissolves rapidly in an aqueous solution. For example, at 20° C., a spray-dried mannitol powder dissolves in water at a rate that is 7 times faster than crystalline mannitol and 20 times faster than granular mannitol. Also, spray-dried mannitol dissolves in water faster than conventional white sugar, white sugar for direct-compression, granular sorbitol and dextrate (a hydrolyzed starch) by factors of 10, 5-9, 7 and 3, respectively (see Test Example 1). In view of the fact that the water-solubilities of the above-mentioned saccharides are about 8 times higher than that of spray-dried mannitol, the markedly high dissolution rate of spray-dried mannitol is remarkable.

A spray-dried mannitol powder has improved flowability and compressibility than conventional crystalline mannitol, and thus, the tablet of the present invention may be obtained by a direct-compress process. Further, the improved compressibility of the spray-dried mannitol allows the hardness control of the resulting tablet through varying the compression pressure. Also, the spray-dried mannitol is sweet (about 0.5 times than white sugar), pleasing to the taste of patients.

The spray-dried mannitol is preferably used in an amount ranging from 30 to 95 wt % based on the total weight of the inventive tablet.

The tablet of the present invention further comprises crospovidone in an amount ranging from 1 to 10 wt % based on the total weight of the tablet as a secondary disintegrant, which enhances the dissolution (disintegration) rate of the spray-dried mannitol by way of bringing water in contact with the spray-dried mannitol through its capillary action.

The tablet of the present invention may also contain one or more pharmaceutically acceptable excipients, including organic acids such as citric acid, tartaric acid, fumaric acid, and malic acid; and effervescent agents such as calcium carbonate, sodium bicarbonate and potassium bicarbonate. The organic acid and effervescent agent may be used in amounts ranging from 1 to 5 wt % based on the total weight of the tablet, respectively.

The organic acids stimulate a salivary grand (parotid grand, sublingual grand, and submaxillary gland) to facilitate saliva secretion, thereby accelerating the disintegration of the tablet, although the disintegration effect of organic acids per se is weak. Further, because the effervescent agent can react with water to give carbon dioxide, in case of using them in the tablet of the present invention, the effervescent agent react with saliva and/or organic acids in the oral cavity to give carbon dioxide, thus reducing the disintegration time of the inventive tablet.

Other pharmaceutically acceptable excipients may be also used in the present invention, including but not limited to: sweetening agents such as aspartam, saccharin, ammonium glycyrrhizinate, xylitol, sorbitol and sucrose; and lubricants such as colloidal silicon dioxide, magnesium stearate and magnesium trisilicate.

The tablet of the present invention disintegrates rapidly in the oral cavity, leaving no significant amount of water-insoluble matter therein, and is not easily friable, as shown in the following Test Examples.

The Examples and Test Examples are given for the purpose of illustration only, and are not intended to limit the scope of the invention.

### EXAMPLE 1

12 g of aspartam and 3 g of colloidal silicon dioxide, each screened through a 20-mesh sieve, were mixed and added thereto were 490.5 g of spray-dried mannitol (Pearlitol SD 200®, Roquette), 18 g of sodium bicarbonate, and 18 g of citric acid, each screened through a 40-mesh sieve. This mixture was further mixed with 30 g of crospovidone powder, screened through a 20-mesh sieve, and then with 12 g of magnesium trisilicate, 4.5 g of strawberry flavor and 12 g of magnesium stearate each screened through a 40-mesh sieve (see Table 1-1).

The resultant mixture was compressed into a tablet, using a single type tableting machine (Manesty F3, Manesty Machine Ltd.), to provide a rapidly disintegrable tablet each weighing 600 mg.

### EXAMPLES 2-6

The procedure of Example 1 was repeated using the components and active ingredients shown in Tables 1-1˜1-3 to obtain tablets according to the present invention.

TABLE 1-1
(Unit: gram)

| | | Ex. 1 | Ex. 2 | Ex. 3 | Ex. 4 | Ex. 5 | Ex. 6 |
|---|---|---|---|---|---|---|---|
| Active | Aacetaminophen | — | 500.0 | — | — | — | — |
| ingredients | Domperidone | — | 10.0 | — | — | — | — |
| | Famotidine | — | — | 20.0 | — | — | — |
| | Meclizine | — | — | — | 25.0 | — | — |
| | hydrochloride | | | | | | |
| | Scopolamine | — | — | — | 0.1 | — | — |
| | hydrobromide | | | | | | |
| | Ondansetron HCl | — | — | — | — | 10.0 | — |
| | Cisapride | — | — | — | — | — | 10.0 |
| Dis-integrants | Spray-dried | 490.5 | 675.3 | 634.0 | 383.6 | 235.0 | 153.0 |
| | mannitol | | | | | | |
| | Crospovidone | 30.0 | 72.5 | 40.0 | 25.0 | 15.0 | 10.0 |
| Organic Acids | Citric acid | 18.0 | 43.5 | 24.0 | 15.0 | 9.0 | 6.0 |
| Effervescent | Sodium | 18.0 | 43.5 | 24.0 | 15.0 | 9.0 | 6.0 |
| agents | bicarbonate | | | | | | |
| Sweetening agents | Aspartam | 12.0 | 29.0 | 16.0 | 10.0 | 6.0 | 4.0 |
| Flavors | Strawberry flavor | 4.5 | 10.9 | 6.0 | 3.8 | 2.5 | 2.0 |
| Lubricants | Colloidal silicon | 3.0 | 7.3 | 4.0 | 2.5 | 1.5 | 1.0 |
| | dioxide | | | | | | |
| | Magnesium | 12.0 | 29.0 | 16.0 | 10.0 | 6.0 | 4.0 |
| | trisilicate | | | | | | |
| | Magnesium | 12.0 | 29.0 | 16.0 | 10.0 | 6.0 | 4.0 |
| | stearate | | | | | | |

| | | | | | | |
|---|---|---|---|---|---|---|
| Total weight | 600 | 1,450 | 800 | 500 | 300 | 200 |
| Pressure scale (gauge) | 16.0 | 29.0 | 19.0 | 18.0 | 17.0 | 14.0 |
| Diameter (mm) | 12.5 | 18.0 | 14.0 | 12.0 | 10.0 | 9.5 |
| Number of tablets | 1,000 | 1,000 | 1,000 | 1,000 | 1,000 | 1,000 |

TABLE 1-2
(Unit: gram)

| | | Ex. 7 | Ex. 8 | Ex. 9 | Ex. 10 |
|---|---|---|---|---|---|
| Active | Aacetaminophen | 500.0 | 325.0 | 160.0 | — |
| ingredients | Granisetron HCl | — | — | — | 1.1 |
| Dis- | Spray-dried | 320.0 | 405.0 | 404.0 | 150.0 |
| integrants | mannitol | | | | |
| | Crospovidone | 94.0 | 94.0 | 72.0 | 16.0 |
| Diluents | Xylitol | 100.0 | 133.0 | 100.0 | 17.0 |
| Organic | Citric acid | 21.0 | 21.0 | 16.0 | 4.0 |
| Acids | | | | | |
| Flavors | Herbal flavor | 10.0 | 30.0 | 24.0 | 4.0 |
| Sweetening | Aspartam | 11.0 | 10.5 | 8.0 | 2.0 |
| agents | | | | | |
| Lubricants | Magnesium | 22.0 | 21.0 | 8.0 | 4.0 |
| | trisilicate | | | | |
| | Magnesium | 22.0 | 10.5 | 8.0 | 1.9 |
| | stearate | | | | |

| | | | | |
|---|---|---|---|---|
| Total weight | 1,100 | 1,050 | 800 | 200 |
| Pressure scale (gauge) | 24.0 | 21.0 | 19.0 | 14.0 |
| Diameter (mm) | 16.0 | 16.0 | 14.0 | 9.5 |
| Number of tablets | 1,000 | 1,000 | 1,000 | 1,000 |

TABLE 1-3
(Unit: gram)

| | | Ex. 11 | Ex. 12 | Ex. 13 |
|---|---|---|---|---|
| Active | Sildenafil | 100.0 | — | — |
| ingredients | Loratadine | — | 10.0 | — |
| | Amlodipine | — | — | 5.0 |
| Dis-integrants | Spray-dried | 460.0 | 186.0 | 205.0 |
| | mannitol | | | |
| | Crospovidone | 72.0 | 18.0 | 20.0 |
| Diluents | Xylitol | 100.0 | 25.0 | — |
| Organic | Citric acid | 16.0 | 5.0 | 5.0 |
| Acids | | | | |
| Flavors | Herbal flavor | 20.0 | 6.0 | 5.0 |
| Sweetening | Aspartam | 8.0 | 2.5 | 2.5 |
| agents | | | | |
| Lubricants | Magnesium | 16.0 | 5.0 | 5.0 |
| | trisilicate | | | |
| | Magnesium | 8.0 | 2.5 | 2.5 |
| | stearate | | | |

| | | | |
|---|---|---|---|
| Total weight | 800 | 260 | 250 |
| Pressure scale (gauge) | 20.0 | 17.0 | 17.0 |
| Diameter (mm) | 14.0 | 10.0 | 10.0 |
| Number of tablets | 1,000 | 1,000 | 1,000 |

### COMPARATIVE EXAMPLES 1-1, 1-2, 1-3 AND 1-4

The procedure of Example 1 was repeated except that dextrate, white sugar A for direct compression, white sugar B for direct compression, and sorbitol were each used in place of the spray-dried mannitol to obtain comparable tablets 1-1, 1-2, 1-3 and 1-4, respectively.

### COMPARATIVE EXAMPLES 2-1, 2-2 AND 2-3

The procedure of Example 2 was repeated except that cross-linked carboxymethyl cellulose, sodium starch glycolate, and low substituted hydroxypropyl cellulose were each used in place of crospovidon to obtain comparable tablets 2-1, 2-2 and 2-3, respectively.

### COMPARATIVE EXAMPLES 3-1˜3-3, 4-1˜4-3, 5-1˜5-3 and 6-1˜6-3

The procedures of Example 3-6 were repeated except that cross-linked carboxymethyl cellulose, sodium starch glycolate, and low substituted hydroxypropyl cellulose were each used in place of crospovidon to obtain respective comparable tablets.

### REFERENCE EXAMPLE

Four sieves each having an opening size of 100 mesh, 120 mesh, 140 mesh and 200 mesh were placed in a test screening machine (a product of SIEMENS), in that order from the top of the machine. 50 g of the spray-dried mannitol used in the Example 1 was placed in the 100-mesh sieve at the top of the test machine, and the machine was shaken at 300 rpm for 5 minutes. The amount of the spray-dried mannitol remaining on each sieve was weighed to calculate the particle size distribution of the spray-dried mannitol. The result is shown in Table 2.

TABLE 2
Particle size distribution of spray-dried mannitol

| Particle Size (Mesh) | | Particle Size (μm) | | Weight (g) | Weight (%) |
|---|---|---|---|---|---|
| Above | 100 | Above | 150 | 35.31 | 70.62 |
| 100˜120 | | 125˜150 | | 5.27 | 10.54 |
| 120˜140 | | 106˜125 | | 4.58 | 9.16 |
| 140˜200 | | 90˜106 | | 4.40 | 8.80 |
| Below | 200 | Below | 90 | 0.44 | 0.88 |

| | | |
|---|---|---|
| Total | 50.00 | 100 |

Table 2 shows that the spray-dried mannitol used in the rapidly disintegrable tablet according to the present invention comprises particles having a size greater than 106 μm in an amount of 90.32 wt %.

The tablets prepared in Examples and Comparative Examples were tested as follows.

### TEST METHOD

The hardness and dissolution time in the oral cavity were measured by the following methods.

(1) Hardness

The hardness of each tablet was measured with a tablet hardness tester (Schleuniger-2E, Dr. K. Schleuniger & Co.). The test was repeated 3-10 times for each sample and the results were averaged.

(2) Dissolution time

The time for a sample to completely disintegrate in the oral cavity of a male adult was measured. The test was duplicated three times and the results were averaged.

### Test Example 1

5 g of each of the test materials as shown in Table 3 was added to 150 ml of purified water at 20° C. The time for the material to completely dissolve was measured and the results are shown in Table 3.

TABLE 3

| Compounds | Time (seconds) |
|---|---|
| Spray-dried mannitol | 5 |
| (Pearlitol SD 200 ®, Roquette) | |
| Dextrate | 16 |
| (Endex ®, Edward Mendell) | |
| White sugar for direct compression A | 25 |
| (Sugartab ®, Edward Mendell) | |
| Crystalline mannitol | 35 |
| Sorbitol | 35 |
| (Neosorb ®, Roquette) | |
| White sugar for direct compression B | 45 |
| (Di-Pac ®, Domino Sugar Co.) | |
| White sugar | 50 |
| Xylitol | 74 |
| (XYLISORB ®, Roquette) | |
| Granular mannitol | 100 |
| (Pearlitol 400 DC ®, Roquette) | |

As can be shown in Table 3, the spray-dried mannitol dissolve more quickly than conventional sugar type excipients in an aqueous medium.

### Test Example 2

The hardnesses and disintegration time in the oral cavity were measured for the tablets obtained in Example 1 and Comparative Examples 1-1 to 1-4. The results are shown in Table 4.

TABLE 4

| | Hardness | Disintegration Time |
|---|---|---|
| | (kp) | (second) |
| Example 1 | 6.0 | 22.0 |
| Comp. Ex. 1-1 | 6.1 | 42.3 |
| Comp. Ex. 1-2 | 6.0 | 59.3 |
| Comp. Ex. 1-3 | 6.1 | 51.7 |
| Comp. Ex. 1-4 | 6.2 | 40.3 |

As can be seen in Table 4, the tablet obtained in Example 1, which contains spray-dried mannitol, disintegrates much faster than the comparable tablets containing conventional sugar type excipients.

### Test Example 3

The hardness and disintegration time in the oral cavity measured for the tablets obtained in Examples and Comparative Examples are shown in Table 5.

TABLE 5

| | Hardness (kp) | Disintegration Time (second) |
|---|---|---|
| Example 2 | 7.1 | 45.0 |
| Comp. Example 2-1 | 5.9 | 60.7 |
| Comp. Example 2-2 | 5.0 | 100.0 |
| Comp. Example 2-3 | 5.1 | 140.3 |
| Example 3 | 6.1 | 35.3 |
| Comp. Example 3-1 | 5.4 | 54.7 |
| Comp. Example 3-2 | 4.9 | 70.0 |
| Comp. Example 3-3 | 4.8 | 97.3 |
| Example 4 | 6.2 | 30.7 |
| Comp. Example 4-1 | 5.4 | 54.7 |
| Comp. Example 4-2 | 5.2 | 79.0 |
| Comp. Example 4-3 | 5.0 | 103.3 |
| Example 5 | 5.1 | 30.0 |
| Comp. Example 5-1 | 4.5 | 50.7 |
| Comp. Example 5-2 | 4.3 | 70.3 |
| Comp. Example 5-3 | 4.6 | 95.0 |
| Example 6 | 4.8 | 23.3 |
| Comp. Example 6-1 | 4.0 | 46.7 |
| Comp. Example 6-2 | 3.9 | 70.0 |
| Comp. Example 6-3 | 4.1 | 91.3 |

The results in Table 5 show that the inventive tablets show much shorter disintegration times and higher hardness values as compared with the tables of the corresponding Comparative Examples.

### Test Example 4

The hardness and disintegration time in the oral cavity measured for the tablets obtained in Example 7˜13 are shown in Table 6.

TABLE 6

| | Hardness (kp) | Disintegration Time (second) |
|---|---|---|
| Example 7 | 5.4 | 42.0 |
| Example 8 | 4.5 | 40.3 |
| Example 9 | 4.5 | 35.7 |
| Example 10 | 4.1 | 20.7 |
| Example 11 | 6.0 | 47.0 |
| Example 12 | 4.0 | 32.0 |
| Example 13 | 4.0 | 30.3 |

As can be seen in Table 6, the tablets of the present invention show disintegration times of less than 50 seconds.

### Test Example 5

This experiment is to illustrate excellent disintegration property of the spray-dried mannitol used in the present invention.

The disintegration time in purified water of round tablets made of the two formulations shown in Table 6 were measured. Each tablet had a hardness of 14 kp and a diameter of 10.0 mm. The results for the test are shown in Table 7.

TABLE 7

| | Inventive | Conventional |
|---|---|---|
| | Composition | Composition |
| Recipe | Spray-dried mannitol | Crystalline mannitol |
| | 49.0 g | 49.0 g |
| | Magnesium stearate | Magnesium stearate |
| | 1.0 g | 1.0 g |
| Disintegration Time | | |
| At 20° C. | 5 min | 30 min |
| At 37° C. | 3 min | 12 min |

Table 7 shows that the tablet containing spray-dried mannitol according to the present invention dissolves much more quickly (about 6 times) than the tablet containing a conventional crystalline mannitol.

### Test Example 6

The disintegration in the oral cavity of round tablets respectively made of the two formulations is shown in Table 8. Each tablet had a hardness of 4.5 kp and adiameter of 10.0 mm. The results for the test are shown in Table 8.

TABLE 8

| | Inventive | Conventional |
|---|---|---|
| | Composition | Composition |
| Recipe | Spray-dried mannitol | Crystalline mannitol |
| | 46.0 g | 46.0 g |
| | Crospovidone 3.0 g | Crospovidone 3.0 g |
| | Magnesium stearate | Magnesium stearate |
| | 1.0 g | 1.0 g |
| Disintegration Time | 55 seconds | 95 seconds |

Table 8 also shows that the tablet containing spray-dried mannitol according to the present invention disintegrates much faster than the tablet containing a conventional crystalline mannitol.

While the invention has been described with respect to the above specific embodiments, it should be recognized that various modifications and changes may be made by those skilled in the art, which also fall within the scope of the invention as defined by the appended claims.

What is claimed is:

1. A tablet for oral administration, which disintegrates in the oral cavity within 60 seconds, consisting essentially of (i) a therapeutically effective amount of an active ingredient, (ii) spray-dried mannitol, of which at least 80% has an average particle size over 100 μm, (iii) crospovidone, and (iv) one or more pharmaceutically acceptable excipients, the tablet containing no microcrystalline cellulose.

2. The tablet of claim 1, wherein the contents of the spray-dried mannitol and the crospovidone are in the ranges of 30 to 95% and 1 to 10% by weight, respectively, based on total weight of the tablet.

3. The tablet of claim 1, wherein the active ingredient is selected from the group consisting of acetaminophen, domperidone, famotidine, meclizine hydrochloride, scopolamine hydrobromide, ondansetron hydrochloride, cisapride, granisetron, sildenafil, loratadine and amlodipine.

4. A process for the preparation of a tablet according to claim 1, comprising direct-compressing a mixture consisting essentially of (i) a therapeutically effective amount of an active ingredient, (ii) spray-dried mannitol, of which at least 80% has an average particle size over 100 μm, (iii) crospovidone, and (iv) one or more pharmaceutically acceptable excipients, the tablet containing no microcrystalline cellulose.
